(19) United States
(12) Patent Application Publication
(10) Pub. No.: US 2020/0008580 A1
(43) Pub. Date: Jan. 9, 2020

(54) LEGLESS CHAIR

(71) Applicant: OPTI CO., LTD., Hiroshima (JP)

(72) Inventor: Fumihiro KANDA, Hiroshima (JP)

(73) Assignee: OPTI CO., LTD., Hiroshima (JP)

(21) Appl. No.: 16/573,151

(22) Filed: Sep. 17, 2019

Related U.S. Application Data
(63) Continuation of application No. PCT/JP2017/011118, filed on Mar. 21, 2017.

(51) Int. Cl. A47C 7/02; A61F 5/01

(52) U.S. Cl. CPC A47C 7/029; A47C 7/021; A61H 2203/0425; A61H 2201/0149; A61F 5/01

(57) ABSTRACT

A legless chair including a seat portion and a back portion, the legless chair comprising: grooves formed to extend in a substantially longitudinal direction, from a front edge toward positions corresponding to a lateral pair of ischial tuberosity at a rear, on a surface of the seat portion, wherein inclined surfaces defining the grooves are surfaces inclined gradually in a lateral direction.

### CROSS REFERENCE TO RELATED APPLICATIONS

This is a continuation application of International Patent Application No. PCT/JP2017/011118 filed on Mar. 21, 2017, of which full contents are incorporated herein by reference.

### BACKGROUND OF THE INVENTION

### Field of the Invention

The present invention relates to a legless chair for correcting dysfunction of the sacroiliac joint interposed between the sacrum and ilium bones of the pelvis.

### Description of the Background Art

In a pregnant body's giving birth, e.g., the ligament of the sacroiliac joint of the body is loosened, and as a result, a gap between the sacrum and ilium bones of the body is broadened. In general, there have been some probabilities that the sacrum and ilium bones with such a broadened gap are not restored to a normal positional relation, which broadens a gap of the sacroiliac joint so as to cause a sacroiliac joint dysfunction. It has been known that a lower back pain is one of the symptoms of sacroiliac joint dysfunction.

It is disclosed, in Patent Document 1, that a correctional device, which includes: a recess part corresponding in shape to the pelvic portion of the body; a raised part formed at a rear of the recess part; and a slightly downward-facing inclined part extending from the raised part in a rearward direction, has been configured such that, when a user places the pelvic portion of the body in the recess part of the device to lie on his or her back, the abdomen portion of the body is held up by the raised part of the device and the back portion of the body is supported by the inclined part of the device.

It is disclosed, in Patent Document 2, that chair equipment for correcting the pelvis, which has a space between left-and-right side surface boards laterally narrowed in a downward direction, has been configured such that a person sitting on the equipment is supported not by a seat surface but by the left-and-right side surface boards.

### PRIOR ART DOCUMENTS

### Patent Documents

Patent Document 1: Japanese Patent Application Publication No. 2006-102256

Patent Document 2: Japanese Patent Application Publication No. 2005-103215

### Problems to be Solved

The invention described in Patent Document 1 has been configured such that the pelvic portion of the body of a user lying on the device is positioned symmetrically with respect to the recess part of the device, and the sacrum bone of the pelvis of the body is pressed longitudinally by the raised part of the device. For this reason, there has been a problem that a laterally-broadened gap of the sacroiliac joint could not be corrected.

The invention described in Patent Document 2 has been configured to apply a lateral pressure to the pelvic and lower back portions of the body of a person sitting on the equipment such that the lateral pressure increases in a downward direction of the body. The ischium of the body is contractedly applied with the highest lateral pressure from both the side surface boards, while an upper side of the sacroiliac joint positioned above the ischium is inversely expanded. For this reason, there has been a problem that a lateral gap of the sacroiliac joint could not be corrected.

### SUMMARY OF THE INVENTION

In view of the above-described problems, there is provided the present invention whose objective is to provide a legless chair capable of reducing a gap generated in a left/right sacroiliac joint broadened and misaligned due to its loosened ligament, thereby to narrow each gap and correct each left/right sacroiliac joint to an appropriate position.

### Means for Solving Problems

A first aspect of the present invention is a legless chair (1) including a seat portion (2) and a back portion (3), the legless chair (1) comprising: grooves (5; 4) formed to extend in a substantially longitudinal (front-to-back) direction, from a front edge toward positions corresponding to a lateral pair of (left and right) ischial tuberosity (15; 15) of a body of a user sitting in the legless chair (1) at a rear, on a surface of the seat portion (2), wherein inclined surfaces (30, 31; 30, 31) defining the grooves (5; 4) are surfaces inclined gradually in a lateral (left-and-right) direction.

Further, a second aspect of the present invention is the legless chair (1), in the above first aspect, characterized in that the inclined surfaces (30, 31; 30, 31) defining the grooves (5; 4) formed at regions on the seat portion (2) corresponding to a lateral pair of (left and right) ischia (17; 16) of the body include: inner inclined surfaces (30; 30) formed, at regions corresponding to the buttocks of the body, such that front inner sides of lower surfaces of the lateral pair of ischia (17; 16) are expanded across said inner inclined surfaces (30; 30); and outer inclined surfaces (31; 31) formed, at regions corresponding to a lateral pair of (left and right) rear ends of the lateral pair of ischia (17; 16), such that the lateral pair of ischia (17; 16) are restricted in moving in a rear-outward direction and lateral rotation of the ischia (17; 16) is induced.

Still further, a third aspect of the present invention is the legless chair (1), in the above first or second aspect, characterized in that the back portion (3) includes pressing portions (7; 7) formed, at regions corresponding in height to a lateral pair of (left and right) the posterior superior iliac spine (13; 13), such that outer side portions of the lateral pair of the posterior superior iliac spine (13; 13) could be pressed from a lateral pair of outer sides toward a lateral pair of inner sides, respectively.

### Advantageous Effects of the Invention

According to the legless chair (1) in the first or second aspect of the present invention, front inner sides of the lower surfaces of the left and right ischia (17; 16) are expanded by the inner inclined surfaces (30; 30), while rear outer sides of the left and right ischia (17; 16) are restricted in moving in a rear-outward direction by the outer inclined surfaces (31; 31) at their respective rear ends of the seat portion (2). As seen from above, therefore, the left and right ischia (17; 16) could be caused to rotate in left and right directions, respectively, and left and right ilia (19; 18) integral with their respective ischia (17; 16) could also be caused to rotate in left and right directions, respectively.

According to such a structure, the left ilium (19) is induced to rotate leftward, while the right ilium (18) is induced to rotate rightward, and as a result, the left and right the posterior superior iliac spine (13; 13) could be brought in closer to each other, which results in a close contact between rear portions of each sacroiliac joint (11).

According to the legless chair (1) in the third aspect of the present invention, the left and right the posterior superior iliac spine (13; 13) are directly pressed inward, and as a result, the left and right the posterior superior iliac spine (13; 13) could be brought in closer to each other, and a gap between each ilium (19; 18) and each sacrum (12; 12) could be further effectively corrected so that the gap could be restored to a normal spacing.

### BRIEF DESCRIPTIONS OF THE DRAWINGS

FIG. 1 depicts a perspective view of a legless chair showing one embodiment according to the present invention;

FIG. 2 depicts a plan view schematically showing another embodiment of a seat portion according to the present invention;

FIG. 3 depicts a plan view schematically showing still another embodiment of a seat portion according to the present invention;

FIG. 4 depicts a front-to-back cross-sectional view as taken along the line A-A showing a laterally-central portion of the seat portion shown in FIG. 1;

FIG. 5 depicts a front-to-back cross-sectional view as taken along the line B-B showing a region, corresponding to a left thigh, of the seat portion shown in FIG. 1;

FIG. 6 depicts a left-to-right cross-sectional view as taken along the line C-C showing a region, corresponding to longitudinally-central portions of both thighs, of the seat portion shown in FIG. 1;

FIG. 7 depicts a left-to-right cross-sectional view as taken along the line D-D showing a region, corresponding to ilia, of the seat portion shown in FIG. 1;

FIG. 8 depicts a left-to-right cross-sectional view as taken along the line E-E showing a region, corresponding to the posterior superior iliac spine, of a back portion shown in FIG. 1;

FIG. 9 depicts an explanatory front view of bones of a pelvis;

FIG. 10 depicts an explanatory perspective view of bones of a pelvis as taken from a front side in an obliquely right direction;

FIG. 11 depicts an explanatory cross-sectional view as taken along the line F-F of a portion of ischial tuberosity shown in FIG. 9;

FIG. 12 depicts an explanatory cross-sectional view as taken along the line K1-K1 of an ischium-contact section shown in FIG. 3 along with a portion of an ischium;

FIG. 13 depicts an external perspective view of the legless chair showing one embodiment according to the present invention;

FIG. 14 is a photograph of the legless chair as taken from above showing another embodiment according to the present invention;

FIG. 15 is a photograph of the legless chair with a pelvis model placed therein as taken from above showing a pelvis state for a movement check in another embodiment according to the present invention; and

FIG. 16 is a photograph of the legless chair with a pelvis model placed therein as taken from the side showing a pelvis state for a movement check in another embodiment according to the present invention.

### DESCRIPTIONS OF EMBODIMENTS OF THE INVENTION

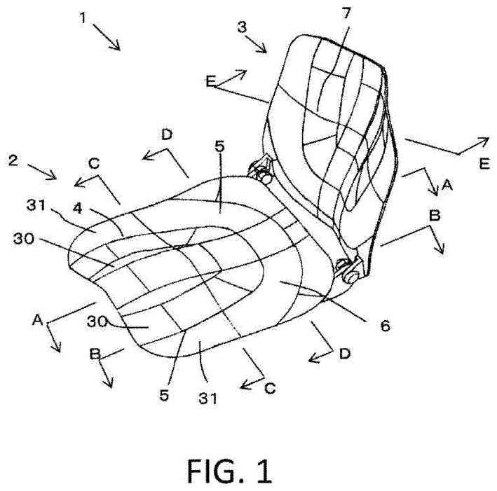

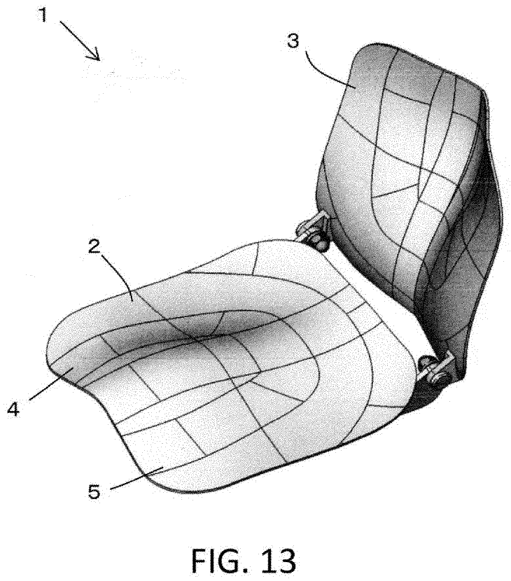

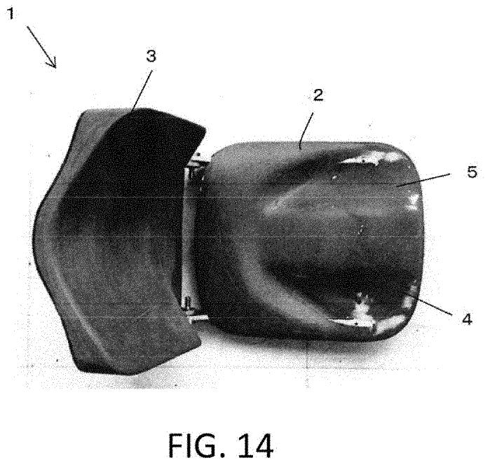

As shown in FIGS. 1, 13 to 16, a legless chair 1 as an embodiment according to the present invention includes a seat portion 2 and a back portion 3. As shown in FIGS. 1 to 3, 6, the seat portion 2 of the legless chair 1 includes grooves 5, 4 formed to extend in a substantially front-to-back direction, on a surface of the seat portion 2, from a front edge of the seat portion 2, toward positions corresponding to left and right ischial tuberosity 15, 15 of the body at a rear on the surface of the seat portion 2, where the grooves 5 and 4 are defined as a pair of inclined surfaces 30, 31 and 30, 31, respectively, and such inclined surfaces 30, 31 are so formed as to be inclined gradually in a left-and-right direction.

The above position corresponding to a left-side ischial tuberosity 15 is defined between an ischium-contact section 60 of an inner inclined surface 30 and an ischium-contact section 61 of an outer inclined surface 31 at a left side.

The above position corresponding to a right-side ischial tuberosity 15 is defined between an ischium-contact section 60 of an inner inclined surface 30 and an ischium-contact section 61 of an outer inclined surface 31 at a right side.

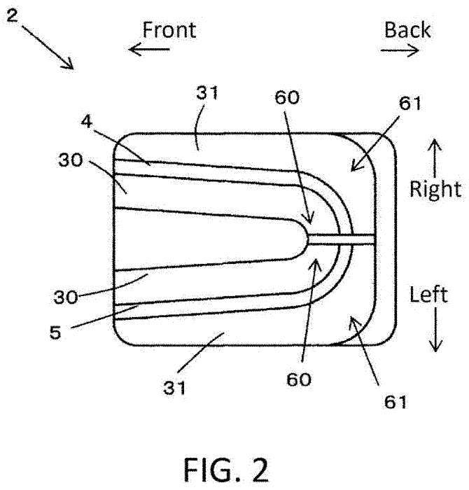

Both of the above-described positions are also regions corresponding to the buttocks of the body as shown in FIGS. 2, 3, 5, 7, 12.

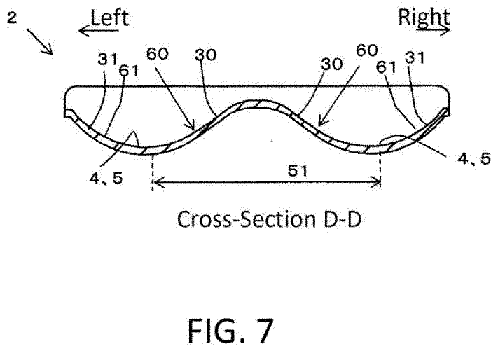

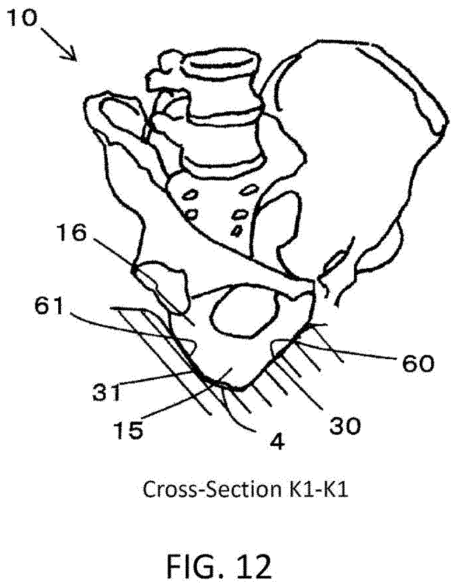

As shown in FIGS. 7, 12, the grooves 5 and 4 are so defined by the left and right inclined surfaces 30, 31 and 30, 31 as to have a range, within which the left and right ischial tuberosity 15 and 15 at a bottom of a pelvis 10 of the body are allowed to fit, respectively, of which:

left and right ischium-contact sections 60, 60, corresponding to an external shape of the buttocks of the body, of the left and right inner inclined surfaces 30, 30 are formed such that front inner sides of lower surfaces of the left and right ischia 17, 16 are expanded, respectively; and

left and right ischium-contact sections 61, 61, corresponding to an external shape of the pelvic portion of the body at regions corresponding to left and right rear ends of the left and right ischia 17, 16, of the left and right outer inclined surfaces 31, 31 are formed such that the left and right ischia 17, 16 are restricted in moving in a rear-outward direction, respectively.

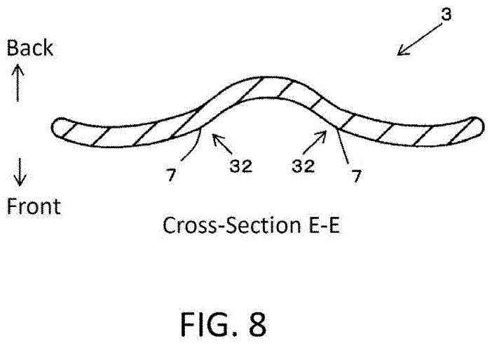

As shown in FIGS. 1, 8, the back portion 3 of the legless chair 1 includes pressing portion 7, 7 formed at regions corresponding to left and right the posterior superior iliac spine 13, 13 of the body. Rear and outer side portions of the left and right the posterior superior iliac spine 13, 13 could be pressed, by the pressing portions 7, 7, from left and right outer sides toward left and right inner sides, respectively.

As shown in FIGS. 1 to 7, the grooves 5, 4 are formed by the left and right inner inclined surfaces 30, 30 and the left and right outer inclined surfaces 31, 31, respectively, in a front-to-back direction from a front edge of the seat portion 2 to the vicinity of a rear edge of the seat portion 2. With respect to the vicinity of a central portion of the grooves 5, 4 along a front-and-back direction, left and right thighs of the body are allowed to fit into the grooves 5, 4 at a front side, and a lower portion of the pelvis 10 is allowed to fit into the grooves 5, 4 at a rear side, respectively. In other words, the left thigh and left ischium 17 are allowed to fit into the groove 5, and the right thigh and right ischium 16 are allowed to fit into the groove 4.

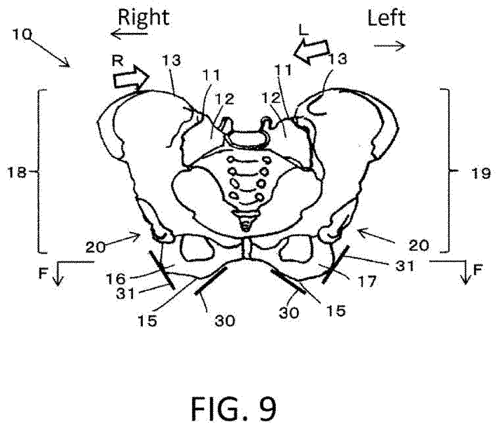

As shown in FIGS. 9 to 12, a distance between the left and right ischial tuberosity 15, 15 is expanded in directions indicated as arrows “b” and “a” by the left and right ischium-contact sections 60, 60 of the inner inclined surfaces 30, 30 corresponding to the buttock to be placed in the left and right grooves 5, 4, respectively, while the left and right ischia 17, 16 are restricted, in moving in a rear-outward direction, by the left and right ischium-contact sections 61, 61 of the outer inclined surfaces 31, 31 corresponding to the left and right rear ends of the left and right ischia 17, 16, respectively.

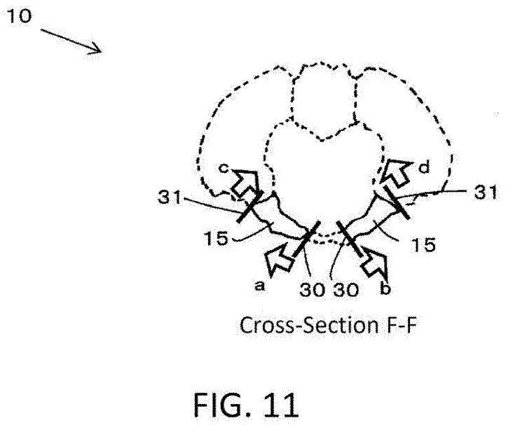

As seen from above in FIG. 11, the left and right ischia 17, 16 could be caused to rotate in left and right directions indicated as the arrows “d” and “c”, respectively, by external forces generated oppositely.

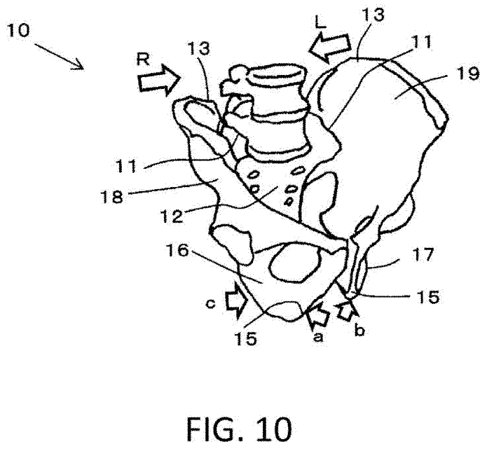

As a result, as seen from above in FIGS. 9, 10, left and right ilia 19, 18 integral with their respective ischia 17, 16 could be caused to rotate in left and right directions indicated as the arrows “L” and “R”, respectively, where the left ilium 19 is induced to rotate leftward, while the right ilium 18 is induced to rotate rightward, and the left and right the posterior superior iliac spine 13, 13 could be brought in closer to each other, and a close contact could occur between rear portions of each of the sacroiliac joints 11, 11, respectively.

As shown in FIGS. 1, 4, 5, in terms of height of the inner inclined surfaces 30, 30 of the grooves 5, 4, a region corresponding to a tailbone of the pelvis 10 is so formed as to be lower in height than the ischium-contact sections 60, 60 corresponding to the ischial tuberosity 15, 15 so that the inner inclined surfaces 30, 30 could be abutted against by the buttock. Further, the groove 5 at a left side and the groove 4 at a right side may be so formed as to communicate with each other without forming the inner inclined surfaces 30, 30 at the region corresponding to the tailbone of the pelvis 10 as long as the buttock is allowed to abut against the inner inclined surfaces 30, 30 and the ischial tuberosity 15, 15 of the pelvis 10 could be expanded laterally.

The grooves 5, 4 are defined by: each width, depth, and orientation; each height and inclination angle of the inner inclined surfaces 30, 30; each height and inclination angle of the outer inclined surfaces 31, 31; or the interval therebetween. As shown in FIG. 12, for values of: the width, depth, orientation of each groove 5, 4; the height and inclination angle of each inner inclined surface 30; the height and inclination angle of each outer inclined surface 31; or the interval between the grooves 5, 4, these values are set such that inner sides of the ischial tuberosity 15, 15 are pressed by the ischium-contact sections 60, 60 of the inner inclined surfaces 30, 30 at the rear of the grooves 5, 4, and thereby, a distance between the left and right ischial tuberosity 15, 15 is expanded, while a rear-outward movement of the left and right ischial tuberosity 15, 15 is restricted by the ischium-contact sections 61, 61 of the outer inclined surfaces 31, 31.

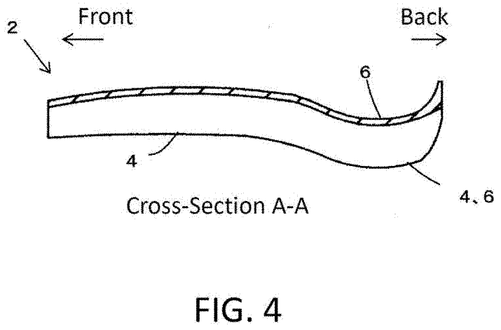

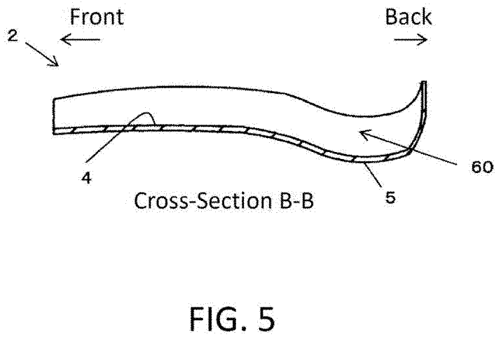

For such proposes, the depth, e.g., of the grooves 5, 4 may be constant from a front edge of the seat portion 2 to the vicinity of a rear edge of the seat portion 2, or may be so deep as to form a recess 6 in the vicinity of the rear edge of the seat portion 2 as shown in FIGS. 4, 5. With respect to the formation of such a recess 6, it is easier to conform the inner inclined surfaces 30, 30 to the shape of the buttock, and it is also easier to fit the ischia 17, 16 securely between the inner inclined surfaces 30, 30 and outer inclined surfaces 31, 31, respectively, which results in facilitating the outward expansion of the ischia 17, 16, and the restriction of rear-outward movement of the ischia 17, 16, provided concurrently with each other by the inner and outer inclined surfaces 30, 31, respectively.

The depth of the left and right grooves 5, 4 is not particularly limited to the above, however, and any depth may be set without forming the recess 6, as long as the inner surfaces of the ischial tuberosity 15, 15 are pressed by the ischium-contact sections 60, 60 of the inner inclined surfaces 30, 30 to expand a distance between the ischial tuberosity 15, 15, as well as, a rear-outward movement of the ischial tuberosity 15, 15 is restricted by the ischium-contact sections 61, 61 of the outer inclined surfaces 31, 31, at the rear of the left and right grooves 5, 4.

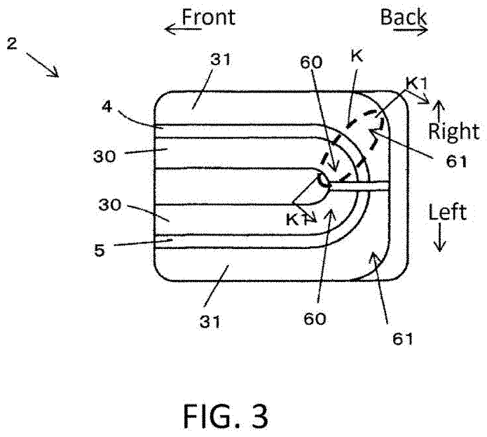

For the orientation of the left and right grooves 5, 4, they may have a shape such as: a substantially V-shape, in a plan view as taken from above in FIG. 2, merging gradually toward a laterally central line from a front to back side of the seat portion 2; a pair of cane shapes inverted to each other with respect to a laterally central line, in a plan view as taken from above in FIG. 3, in substantially parallel to each other from a front to back side, while curving closer to each other around a point corresponding to a tailbone of a pelvic portion; or a U-shape taken as a result of combining the left and right grooves 5, 4 together at an inflection point corresponding to the tailbone of the pelvic portion.

In any shape of the above, the orientation of the left and right grooves 5, 4 is configured such that a region corresponding to the tailbone lower in height than a region corresponding to the buttock, of the inner inclined surfaces 30, 30, is prevented from riding up on ridges of the inner inclined surfaces 30, 30 so as not to abut against the inner inclined surfaces 30, 30 without the ischial tuberosity (15, 15)'s moving down.

As shown in FIGS. 1 to 3, regions in the vicinity of the ischium-contact sections 60, 60 of the inner inclined surfaces 30, 30 and the ischium-contact sections 61, 61 of the outer inclined surfaces 31, 31, in a plan view as taken from above, are formed into arc shapes so that inner sides of the ischial tuberosity 15, 15 could be pressed by the ischium-contact sections 60, 60 of the inner inclined surfaces 30, 30 to be easily expanded in distance between the left and right ischial tuberosity 15, 15. Further, as shown in FIGS. 1, 2, if an interval between the grooves 5, 4 is gradually made narrower from the front end of the seat portion 2 toward the rear of the seat portion 2, a user is supposed to sit on the seat portion 2, while taking such a posture that a distance between his or her left and right thighs is expanded. As a result, the hip joint sections 20 at the bases of the thighs in the pelvis 10 of the user are also pressed outward, which results in outward expanding the left and right ilia 19, 18 and ischia 17, 16, and also in expanding a distance between the left and right ischial tuberosity 15, 15 positioned in the buttock.

There is no particular need, however, to limit the orientation of the grooves 5, 4 such that an interval between the left and right grooves 5, 4 gradually narrows at the rear. The orientation of the grooves 5, 4 is not particularly limited as long as inner sides of the ischial tuberosity 15, 15 are pressed by the ischium-contact sections 60, 60 of the inner inclined surfaces 30, 30 at the rear of the left and right grooves 5, 4 to be expanded in distance between the left and right ischial tuberosity 15, 15, while are prevented by the outer inclined surfaces 31, 31 from being expanded in a rear-outward direction.

For the width of the left and right grooves 5, 4, the grooves 5, 4 at the rear of the seat portion 2 on which a user places the pelvic portion of the body, in particular, should have the width enough to such an extent that the left and right ischia 17, 16 could be fit firmly into the grooves 5, 4, respectively, and such a width should not be too broad or too narrow in comparison with each thickness of the ischia 17, 16.

Other aspects of the grooves 5, 4 are not limited, in particular, and may be configured such that inner sides of the ischial tuberosity 15, 15 are pressed by the ischium-contact sections 60, 60 of the inner inclined surfaces 30, 30 at the rear of the left and right grooves 5, 4 to be expanded in distance between the left and right ischial tuberosity 15, 15, while the ischial tuberosity 15, 15 are restricted by the ischium-contact sections 61, 61 of the outer inclined surfaces 31, 31 in a rear-outward movement.

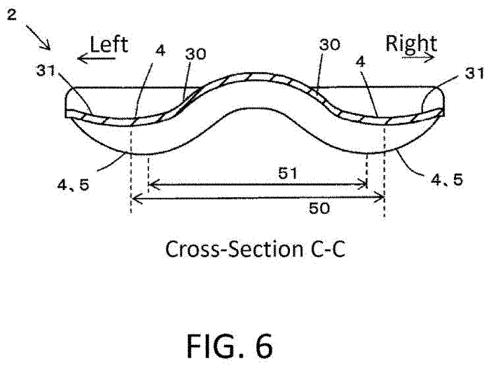

Structural and functional examples of the seat portion 2 are described. As shown in FIGS. 6, 7, a lateral (left-and-right) interval 50 between the left and right grooves 5, 4 at a front region of the seat portion 2 is broader than a lateral interval 51 between the left and right grooves 5, 4 at regions corresponding to the ischial tuberosity 15, 15 at the rear of the seat portion 2. As a result, the left and right thighs are expanded outward and the buttock is expanded outward, and therefore, the left and right ischia 17, 16 are also caused to move outward in left and right directions, respectively. Further, the inclined surfaces 31, 31 at rear-outward sides of the grooves 5, 4 prevent rear outer sides of lower surfaces of the ischial tuberosity 15, 15 from moving outward. When such two events occur coincidentally, the left ischium 17 is caused to rotate leftward, while the right ischium 16 is caused to rotate rightward, and a leftward and rightward rotation is also induced for the ilia 19, 18 so located above as to be integral with the ischia 17, 16, respectively. Such rotation of the ilia 19, 18 causes rear portions of each of the sacroiliac joints 11, 11 to move closer to each other, and as a result, the left and right the posterior superior iliac spine 13, 13 move to narrow a distance therebetween. As a result, a gap between each ilium 19, 18 and each sacrum 12 is narrowed so that a gap broadened and misaligned in each sacroiliac joint 11 due to its loosened ligament could be narrowed and restored to a normal spacing.

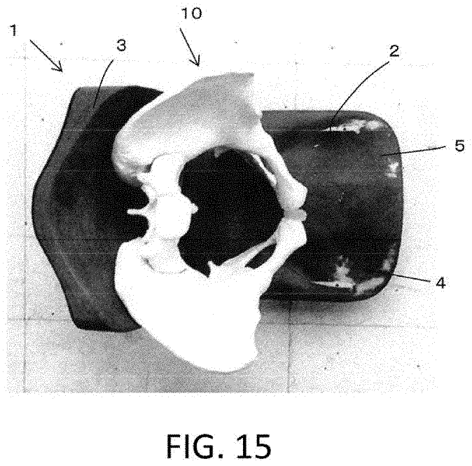

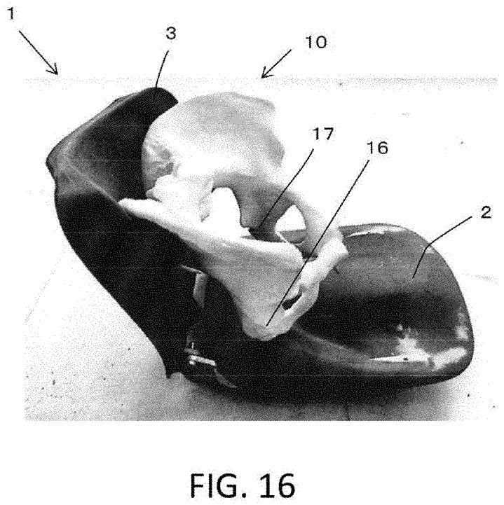

As shown in FIGS. 15, 16 that are photographs of the state where a model of the pelvis 10 is placed in the legless chair 1, the ischial tuberosity 15, 15 as the lowest portion of the pelvis 10 are fit into the grooves 5, 4, respectively. After having confirmed the effects achieved by an embodiment according to the present invention, it has been found that a movement of the right ischium 17 and the left ischium 16 causes the ilium 19, 18 to move so that the left and right the posterior superior iliac spine 13, 13 could move to narrow a distance therebetween. As a result, gaps between the ilia 19, 18 and the sacra 12, 12 are narrowed so that the gaps broadened and misaligned in the sacroiliac joints 11, 11 due to loosened ligaments could be narrowed and restored to normal positional relations, respectively.

The left and right grooves 5, 4, the left and right ischium-contact sections 60, 60 of the inner inclined surfaces 30, 30, and the left and right ischium-contact sections 61, 61 of the outer inclined surfaces 31, 31 could cause the left and right the posterior superior iliac spine 13, 13 to move closer together so as to narrow a gap between each ilium 19, 18 and each sacrum 12, and a gap in each sacroiliac joint 11 broadened and misaligned due to its loosened ligament, thereby capable of correcting each sacroiliac joint 11 to a normal positional relation.

As shown in FIGS. 1, 8, the back portion 3 includes the pressing portions 7, 7 formed, at regions corresponding in height to the left and right the posterior superior iliac spine 13, 13, such that outer side portions of the left and right the posterior superior iliac spine 13, 13 could be pressed from left and right outer sides toward left and right inner sides, respectively.

The pressing portions 7, 7 could press directly the left and right the posterior superior iliac spine 13, 13 in an inward direction so as to narrow a gap between each of the posterior superior iliac spine 13, 13 and each sacrum 12, and a gap in each sacroiliac joint 11 broadened and misaligned due to its loosened ligament, thereby capable of correcting each sacroiliac joint 11 to a normal positional relation.

The method of use is described as follows. A user wearing thin clothing sits in the legless chair 1 as an embodiment according to the present invention in such a manner that the user's left and right ischial tuberosity 15, 15 in his or her buttock are aligned with the left and right ischium-contact sections 60, 60 of the inner inclined surfaces 30, 30, respectively, on the seat portion 2, and the user's left and right the posterior superior iliac spine 13, 13 are pressed by the pressing portions 7, 7, respectively, on the back portion 3. As a result, a gap between the user's the posterior superior iliac spine 13 and the user's sacrum 12 is narrowed, and a gap in the user's sacroiliac joint 11 having been broadened due to the user's loosened ligaments could be narrowed and restored to a normal spacing.

### REFERENCE NUMERALS

1 legless chair
2 seat portion
3 back portion
4 groove
5 groove
6 recess
7 pressing portion
10 pelvis
11 sacroiliac joint
12 sacrum
13 the posterior superior iliac spine
15 ischial tuberosity
16 ischium
17 ischium
18 ilium
19 ilium
20 hip joint section
30 inclined surface
31 inclined surface
50 interval
51 interval
60 ischium-contact section
61 ischium-contact section

What is claimed is:

1. A legless chair including a seat portion and a back portion, the legless chair comprising:
grooves formed to extend in a substantially longitudinal direction, from a front edge toward positions corresponding to a lateral pair of ischial tuberosity at a rear, on a surface of the seat portion,
wherein inclined surfaces defining the grooves are surfaces inclined gradually in a lateral direction.

2. The legless chair according to claim 1, wherein
the inclined surfaces defining the grooves formed at regions on the seat portion corresponding to a lateral pair of ischia include:
inner inclined surfaces formed, at regions corresponding to the buttocks, such that front inner sides of lower surfaces of the lateral pair of ischia are expanded across said inner inclined surfaces; and
outer inclined surfaces formed, at regions corresponding to rear ends of the lateral pair of ischia, such that the lateral pair of ischia are restricted in moving in a rear-outward direction and lateral rotation of the ischia is induced.

3. The legless chair according to claim 1, wherein
the back portion includes
pressing portions formed, at regions corresponding in height to a lateral pair of the posterior superior iliac spine, such that outer side portions of the lateral pair of the posterior superior iliac spine could be pressed from a lateral pair of outer sides toward a lateral pair of inner sides, respectively.

4. The legless chair according to claim 2, wherein
the back portion includes
pressing portions formed, at regions corresponding in height to a lateral pair of the posterior superior iliac spine, such that outer side portions of the lateral pair of the posterior superior iliac spine could be pressed from a lateral pair of outer sides toward a lateral pair of inner sides, respectively.
